(19) United States
(12) Patent Application Publication
(10) Pub. No.: US 2024/0024446 A1
(43) Pub. Date: Jan. 25, 2024

(54) CONJUGATED AFLATOXIN B TO PROTECT AGAINST MYCOTOXICOSIS

(71) Applicant: Intervet Inc., Madison, NJ (US)

(72) Inventors: Maarten Hendrik Witvliet, Oostrum (NL); Ruud Philip Antoon Maria Segers, Boxmeer (NL); Mateusz Walczak, Ottersum (NL)

(73) Assignee: Intervet Inc., Madison, NJ (US)

(21) Appl. No.: 18/257,600

(22) PCT Filed: Dec. 21, 2021
(86) PCT No.: PCT/EP2021/086953

§ 371 Date: Jun. 15, 2023

(30) Foreign Application Priority Data
Dec. 22, 2020 (EP) 20216357.2

(51) Int. Cl. A61K 39/00; A61P 39/00

(52) U.S. Cl. CPC A61K 39/0013; A61P 39/00; A61K 2039/6081

(57) ABSTRACT

The present invention pertains to the use of conjugated aflatoxin (AFB) in a method to protect an animal against AFB induced mycotoxicosis, in particular to protect against a decrease in average daily weight gain, immune suppression, icterus, hemorrhagic enteritis as a result of the ingestion of AFB.

### BACKGROUND OF THE INVENTION

The invention in general pertains to protection of animals against mycotoxicosis induced by mycotoxins. In particular, the invention pertains to protection against mycotoxicosis induced by aflatoxin B (AFB). Aflatoxins produced by the Aspergillus species are highly toxic, carcinogenic, and cause severe contamination to food sources, leading to serious health consequences. Contaminations by aflatoxins have been reported in food and feed, such as groundnuts, millet, sesame seeds, maize, wheat, rice, fig, spices and cocoa due to fungal infection during pre- and post-harvest conditions. Besides these food products, commercial products like peanut butter, cooking oil and cosmetics have also been reported to be contaminated by aflatoxins. Even a low concentration of aflatoxins is hazardous for human and livestock. The ingestion of aflatoxins (AFs) from contaminated food and feed has led to serious health complications in humans and animals. Therefore, different countries have implemented strict regulations for AFs in food and feed to maintain the health of individuals. Besides this, various innovative technologies and control strategies are applied for pre- and post-harvest management of AFs to enhance sustainable agricultural productivity.

Aflatoxins are chemically difuranocoumarin derivatives with a bifuran group attached to the coumarin nucleus and a pentanone ring (in case of AFBs) or a lactone ring (in case of AFGs). AFB's are generally more toxic than AFGs. The two major AFBs among the identified 20 AFs are the closely related AFB1 and AFB2. In particular, AFB1 is a potent carcinogen to humans, and is associated with serious health complications after ingestion of AFB contaminated food. It has been a causal factor for liver cancer and acute hepatitis as well as periodic outbreaks of acute aflatoxicosis leading to death. This explains why most prophylactic treatment of AFB induced mycotoxicosis is currently restricted to good agricultural practice to reduce mycotoxins production on crop and control programs of food and feed commodities to ensure that mycotoxin levels remain below certain limits, and by passive protection by providing anti-AFB antibodies, for example by providing these in milk that potentially contains AFB.

However, AFB also induces disease in all sorts of livestock animals, although dairy and beef cattle seem to be somewhat are more susceptible to aflatoxine induced mycotoxicosis (also denoted as aflatoxicosis) than other species such as swine and poultry. Young animals of all species are more susceptible to the effects of aflatoxins than mature animals. Pregnant and growing animals are less susceptible than young animals but more susceptible than nature animals. Toxicity due to AFBs, under natural conditions, is usually sub-acute or chronic, depending on the level of exposure. Occasionally, acute cases are also seen. In general, affected animals show reduced growth rate, weight loss, immune suppression, icterus, hemorrhagic enteritis, reduced performance, and ultimately death.

Fungi in general cause a broad range of diseases in animals, involving parasitism of organs and tissues as well as allergenic manifestations. However, other than poisoning through ingestion of non-edible mushrooms, fungi can produce mycotoxins and organic chemicals that are responsible for various toxic effects referred to as mycotoxicosis. This disease is caused by exposure to mycotoxins, pharmacologically active compounds produced by filamentous fungi contaminating foodstuffs or animal feeds. Mycotoxins are secondary metabolites not critical to fungal physiology, that are extremely toxic in minimum concentrations to vertebrates upon ingestion, inhalation or skin contact. About 400 mycotoxins are currently recognized, subdivided in families of chemically related molecules with similar biological and structural properties. Of these, approximately a dozen groups regularly receive attention as threats to animal health. Examples of mycotoxins of greatest public interest and agroeconomic significance include aflatoxins (AF), ochratoxins (OT), trichothecenes (T; including deoxynivalenol, abbreviated DON), zearalenone (ZEA), fumonisin (F), tremorgenic toxins, and ergot alkaloids. Mycotoxins have been related to acute and chronic diseases, with biological effects that vary mainly according to the diversity in their chemical structure, but also with regard to biological, nutritional and environmental factors. The pathophysiology of mycotoxicosis is the consequence of interactions of mycotoxins with functional molecules and organelles in the animal cell, which may result in carcinogenicity, genotoxicity, inhibition of protein synthesis, immunosuppression, dermal irritation, and other metabolic perturbations. In sensitive animal species, mycotoxins may elicit complicated and overlapping toxic effects. Mycotoxicosis are not contagious, nor is there significant stimulation of the immune system. Treatment with drugs or antibiotics has little or no effect on the course of the disease. To date no human or animal vaccine is available for combating mycotoxicosis.

A growing body of work is thus focusing in developing vaccines and/or immunotherapy with efficacy against broad fungal classes as a powerful tool in combating mycoses, i.e. the infection with the fungi as such, instead of the toxins, in the prevention of specific fungal diseases. In contrast to mycoses, mycotoxicosis do not need the involvement of the toxin producing fungus and are considered as abiotic hazards, although with biotic origin. In this sense, mycotoxicosis have been considered examples of poisoning by natural means, and protective strategies have essentially focused on exposure prevention. Human and animal exposure occurs mainly from ingestion of the mycotoxins in plant-based food. Metabolism of ingested mycotoxins could result in accumulation in different organs or tissues; mycotoxins can thus enter into the human food chain through animal meat, milk, or eggs (carry over). Because toxigenic fungi contaminate several kinds of crops for human and animal consumption, mycotoxins may be present in all kinds of raw agricultural materials, commodities and beverages. The Food and Agriculture Organization (FAO) estimated that 25% of the world's food crops are significantly contaminated with mycotoxins. At the moment, the best strategies for mycotoxicosis prevention include good agricultural practice to reduce mycotoxins production on crop and control programs of food and feed commodities to ensure that mycotoxin levels stand below predetermined threshold limits. These strategies may limit the problem of contamination of commodities with some groups of mycotoxins with high costs and variable effectiveness. Except for supportive therapy (e.g., diet, hydration), there are almost no treatments for mycotoxin exposure and antidotes for mycotoxins are generally not available, although in individual exposed to AFs some encouraging results have been obtained with some protective agents such as chlorophyllin, green tea polyphenols and dithiolethiones (oltipraz).

In the art, particular vaccination strategies have been proposed against some mycotoxins, mainly to prevent mycotoxicosis by contamination of important foods of animal origin with a strategy based on the production of antibodies that could specifically block initial absorption or bioactivation of mycotoxins, their toxicity and/or secretion in animal products (such as milk) by immuno-interception, directed mainly at preventing mycotoxicosis in humans.

The production of vaccines for protection against mycotoxicosis however are very challenging, principally related to the fact that the mycotoxins themselves are small non-immunogenic molecules, and the toxicity associated with mycotoxins which makes the use as antigens in healthy subjects not risk free. Mycotoxins are low molecular weight, usually non-proteinaceous molecules, which are not ordinarily immunogenic (haptens), but can potentially elicit an immune response when attached to a large carrier molecule such as a protein. Methods for conjugation of mycotoxins to protein or polypeptide carrier and optimization of conditions for animal immunization have been extensively studied, with the purpose of producing monoclonal or polyclonal antibodies with different specificities to be used in immunoassay for screening of mycotoxins in products destined for animal and human consumption. Coupling proteins used in these studies included bovine serum albumin (BSA), keyhole limpet haemocyanin (KLH), thyroglobulin (TG) and polylysine, among others. In the past decades, many efforts have been made for developing mycotoxin derivatives that can be bound to proteins while retaining enough of the original structure so that antibodies produced will recognize the native toxin. Through these methods, antibodies against many mycotoxins have been made available, demonstrating that conjugation to proteins may be an effective tool for the raise of antibodies. The application of this strategy for human and animal vaccination, thus to arrive at protection while being safe for the recipient, has not been successful so far due to the toxic properties of the molecules that might be released in vivo. For example, conjugation of toxins such as T-2 to protein carriers has been shown to result in unstable complexes with potential release of the free toxin in its active form (Chanh et al, Monoclonal anti-idiotype induces protection against the cytotoxicity of the trichothecene mycotoxin T-2, in J Immunol. 1990, 144: 4721-4728). In analogy with toxoid vaccines, which may confer a state of protection against the pathological effects of bacterial toxins, a reasonable approach to the development of vaccines against mycotoxin may be based on conjugated “mycotoxoids”, defined as modified form of mycotoxins, devoid of toxicity although maintaining antigenicity (Giovati L et al, Anaflatoxin B1 as the paradigm of a new class of vaccines based on “Mycotoxoids”, in Ann Vaccines Immunization 2(1): 1010, 2015). Given the non-proteinaceous nature of mycotoxins, the approach for conversion to mycotoxoids should rely on chemical derivatization. The introduction of specific groups in strategic positions of the related parent mycotoxin may lead to formation of molecules with different physicochemical characteristics, but still able to induce antibodies with sufficient cross-reacting to the native toxin. The common rationale for mycotoxin vaccination would thus be based on generating antibodies against the mycotoxoid with an enhanced ability to bind native mycotoxin compared with cellular targets, neutralizing the toxin and preventing disease development in the event of exposure. A potential application of this strategy has been demonstrated in the case of mycotoxins belonging to the AF group (Giovati et al, 2015), but not for any of the other mycotoxins. Moreover, the protective effect has not been demonstrated against mycotoxicosis of the vaccinated animal as such, but only against carry over in dairy cows to their milk, so as to protect people that consume the milk or products made thereof from mycotoxicosis.

### OBJECT OF THE INVENTION

It is an object of the invention to provide a method to actively protect an animal against mycotoxicosis induced by aflatoxin, an important mycotoxin in animal feed.

### SUMMARY OF THE INVENTION

In order to meet the object of the invention it has been found that conjugated aflatoxin (AFB) is suitable for use in a method to actively protect an animal against AFB induced mycotoxicosis. It was found that there was no need to convert the AFB into a toxoid, the conjugated toxin appeared to be safe for the treated host animal. Also, it was surprising to see that an immune response induced against a small molecule such as a mycotoxin is, is strong enough to protect the animal itself against mycotoxicosis after ingestion of the mycotoxin post treatment. Such actual protection of an animal by inducing in that animal an active immune response against a mycotoxin itself has not been shown in the art for any mycotoxin.

### Definitions

Mycotoxicosis is the disease resulting from exposure to a mycotoxin. The clinical signs, target organs, and outcome depend on the intrinsic toxic features of the mycotoxin and the quantity and length of exposure, as well as the health status of the exposed animal.

To protect against mycotoxicosis means to prevent or decrease one or more of the negative physiological effects of the mycotoxin in the animal, such as a decrease in average daily weight gain.

The term Aflatoxin B denotes aflatoxins that are chemically difuranocoumarin derivatives with a bifuran group attached to the coumarin nucleus and a pentanone ring.

In particular the term covers AFB1 and AFB2. The chemical formula of AFB1 is C₁₇H₁₂O₆(cas no 1162-65-8) and that of AFB2 is C₁₇H₁₄O₆(cas no 7220-81-7), having no double bond in the bifuran group. The structural formula of AFB1 is given here below.

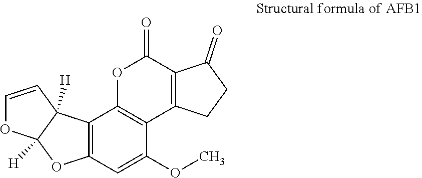 [SMILES: COc1cc2c(c3oc(=O)c4c(c13)CCC4=O)[C@@H]1C=CO[C@@H]1O2]

A conjugated molecule is a molecule to which an immunogenic compound is coupled through a covalent bond. Typically, the immunogenic compound is a large protein such as KLH, BSA or OVA.

An adjuvant is a non-specific immunostimulating agent. In principal, each substance that is able to favor or amplify a particular process in the cascade of immunological events, ultimately leading to a better immunological response (i.e. the integrated bodily response to an antigen, in particular one mediated by lymphocytes and typically involving recognition of antigens by specific antibodies or previously sensitized lymphocytes), can be defined as an adjuvant. An adjuvant is in general not required for the said particular process to occur, but merely favors or amplifies the said process. Adjuvants in general can be classified according to the immunological events they induce. The first class, comprising i.a. ISCOM's (immunostimulating complexes), saponins (or fractions and derivatives thereof such as Quil A), aluminum hydroxide, liposomes, cochleates, polylactic/glycolic acid, facilitates the antigen uptake, transport and presentation by APC's (antigen presenting cells). The second class, comprising i.a. oil emulsions (either W/O, O/W, W/O/W or O/W/O), gels, polymer microspheres (Carbopol), non-ionic block coplymers and most probably also aluminum hydroxide, provide for a depot effect. The third class, comprising i.a. CpG-rich motifs, monophosphoryl lipid A, mycobacteria (muramyl dipeptide), yeast extracts, cholera toxin, is based on the recognition of conserved microbial structures, so called pathogen associated microbial patterns (PAMPs), defined as signal 0. The fourth class, comprising i.a. oil emulsion surface active agents, aluminum hydroxide, hypoxia, is based on stimulating the distinguishing capacity of the immune system between dangerous and harmless (which need not be the same as self and non-self). The fifth class, comprising i.a. cytokines, is based on upregulation of costimulatory molecules, signal 2, on APCs.

A vaccine is in the sense of this invention is a constitution suitable for application to an animal, comprising one or more antigens in an immunologically effective amount (i.e. capable of stimulating the immune system of the target animal sufficiently to at least reduce the negative effects of a challenge with a disease inducing agent, typically combined with a pharmaceutically acceptable carrier (i.e. a biocompatible medium, viz. a medium that after administration does not induce significant adverse reactions in the subject animal, capable of presenting the antigen to the immune system of the host animal after administration of the vaccine) such as a liquid containing water and/or any other biocompatible solvent or a solid carrier such as commonly used to obtain freeze-dried vaccines (based on sugars and/or proteins), optionally comprising immunostimulating agents (adjuvants), which upon administration to the animal induces an immune response for treating a disease or disorder, i.e. aiding in preventing, ameliorating or curing the disease or disorder.

Active protection induced by a vaccine is protection of a vaccinated subject itself by induction of antibodies by the vaccine in the subject, which antibodies protect the subject against a later challenge with the corresponding disease causing pathogen or compound. Active protection is opposed to passive protection whereby a subject animal receives ready-made antibodies produced outside of its body (e.g. in a laboratory animal, or by a mother animal, or recombinantly), in order to protect against the corresponding disease causing pathogen or compound.

### FURTHER EMBODIMENTS OF THE INVENTION

In a further embodiment of the invention, the conjugated AFB is systemically administered to the animal. Although local administration, for example via mucosal tissue in the gastro-intestinal tract (oral or anal cavity) or in the eyes (for example when immunising chickens) is known to be an effective route to induce an immune response in various animals, it was found that systemic administration leads to an adequate immune response for protecting animals against a AFB induced mycotoxicosis. It was found in particular that effective immunisation can be obtained upon intramuscular, oral and/or intradermal administration.

The age of administration is not critical, although it is preferred that the administration takes place before the animal is able to ingest feed contaminated with substantial amounts of AFB. Hence a preferred age at the time of administration of 6 weeks or younger. Further preferred is an age of 4 weeks or younger, such as for example an age of 1-3 weeks.

In yet another embodiment of the invention the conjugated AFB is administered to the animal at least twice. Although many animals (in particular swine chickens, ruminants) in general are susceptible for immunisation by only one shot of an immunogenic composition, it is believed that for economic viable protection against AFB two shots are preferred. This is because in practice the immune system of the animals will not be triggered to produce anti-AFB antibodies by natural exposure to AFB, simply because naturally occurring AFB is not immunogenic. So, the immune system of the animals is completely dependent on the administration of the conjugated AFB. The time between the two shots of the conjugated AFB can be anything between 1 week and 1-2 years. For young animals it is believed that a regime of a prime immunisation, for example at 1-3 weeks of age, followed by a booster administration 1-4 weeks later, typically 1-3 weeks later, such as 2 weeks later, will suffice. Older animals may need a booster administration every few months (such as 4, 5, 6 months after the last administration), or on a yearly or biannual basis as is known form other commercially applied immunisation regimes for animals.

In still another embodiment the conjugated AFB is used in a composition comprising an adjuvant in addition to the conjugated AFB. An adjuvant may be used if the conjugate on itself is not able to induce an immune response to obtain a predetermined level of protection. Although conjugate molecules are known that are able to sufficiently stimulate the immune system without an additional adjuvant, such as KLH or BSA, it may be advantageous to use an additional adjuvant. This could take away the need for a booster administration or prolong the interval for the administration thereof. All depends on the level of protection needed in a specific situation. A type of adjuvant that was shown to be able and induce a good immune response against AFB when using conjugated-AFB as immunogen is an emulsion of water and oil, such as for example a water-in-oil emulsion or an oil-in-water emulsion. The former is typically used in poultry while the latter is typically used in animals who are more prone to adjuvant induced site reactions such as swine and ruminants.

In again another embodiment the conjugated AFB comprises AFB conjugated to a protein having a molecular mass above 10.000 Da. Such proteins, in particular keyhole limpet hemocyanin (KLH) and ovalbumin (OVA), have been found to be able and induce an adequate immune response in animals, in particular in swine and chickens. A practical upper limit for the protein might be 100 MDa.

Regarding the protection against mycotoxicosis, it was found in particular that using the invention, the animal is believed to be protected against a decrease in average daily weight gain, immune suppression, icterus, hemorrhagic enteritis as a result of the ingestion of AFB, thus one or more of these signs of mycotoxicosis induced by AFB.

The invention will now be further explained using the following examples.

### EXAMPLES OF THE INVENTION

In a first series of experiments (see Examples 1-4) it was assessed whether an active immune response against a mycotoxin can be elicited using a conjugated mycotoxin, and if so, is able to protect the vaccinated animal against a disorder induced by this mycotoxin after ingestion thereof. For the latter a pig model for challenge with DON was used. Thereafter (Examples 5 and 6) it was assessed whether or not the use of conjugated AFB in a vaccine can induce antibodies against aflatoxin in the vaccinated animal.

### Example 1: Immunisation Challenge Experiment Using Conjugated DON

Objective

The objective of this study was to evaluate the efficacy of conjugated deoxynivalenol to protect an animal against mycotoxicosis due to DON ingestion. To examine this, pigs were immunised twice with DON-KLH before being challenged with toxic DON. Different routes of immunisation were used to study the influence of the route of administration.

Study Design

Fourty 1 week old pigs derived from 8 sows were used in the study, divided over 5 groups. Twenty-four piglets of group 1-3 were immunised twice at 1 and 3 weeks of age. Group 1 was immunised intramuscularly (IM) at both ages. Group 2 received an IM injection at one week of age and an oral boost at three weeks of age. Group 3 was immunised intradermally (ID) two times. From 5% weeks of age groups 1-3 were challenged during 4 weeks with DON administered orally in a liquid. Group 4 was not immunised but was only challenged with DON as described for groups 1-3. Group 5 served as a control and only received a control fluid, from the age of 5.5 weeks for 4 weeks.

The DON concentration in the liquid formulation corresponded to an amount of 5.4 mg/kg feed. This corresponds to an average amount of 2.5 mg DON per day. After four weeks of challenge all animals were post-mortem investigated, with special attentions for the liver, kidneys and the stomach. In addition, blood sampling was done at day 0, 34, 41, 49, 55, 64 (after euthanasia) of the study, except for group 5 of which blood samples were taken only at day 0, 34, 49, and directly after euthanasia.

Test Articles

Three different immunogenic compositions were formulated, namely Test Article 1 comprising DON-KLH at 50 μg/ml in an oil-in-water emulsion for injection (X-solve 50, MSD AH, Boxmeer) which was used for IM immunization; Test Article 2 comprising DON-KLH at 50 μg/ml in a water-in-oil emulsion (GNE, MSD AH, Boxmeer) which was used for oral immunization and Test Article 3 comprising DON-KLH at 500 μg/ml in an oil-in-water emulsion for injection (X-solve 50) for ID immunisation.

The challenge deoxynivalenol (obtained from Fermentek, Israel) was diluted in 100% methanol at a final concentration of 100 mg/ml and stored at <−15° C. Prior to usage, DON was further diluted and supplied in a treat for administration.

Inclusion Criteria

Only healthy animals were used. In order to exclude unhealthy animals, all animals were examined before the start of the study for their general physical appearance and absence of clinical abnormalities or disease. Per group piglets from different sows were used. In everyday practice all animals will be immunised even when pre-exposed to DON via intake of DON contaminated feed. Since DON as such does not raise an immune response, it is believed that there is no principle difference between animals pre-exposed to DON and naïve with respect to DON.

Results

None of the animals had negative effects associated with the immunisation with DON-KLH. The composition thus appeared to be safe.

All pigs were serologically negative for titres against DON at the start of the experiment, During the challenge the groups immunised intramuscular (Group 1) and intradermally (Group 3) developed antibody responses against DON as measured by ELISA with native DON-BSA as the coating antigen. Table 1 depicts the average IgG values on 4 time points during the study with their SD values. Both Intramuscular immunisation and Intradermal immunisation induced significant titres against DON.

TABLE 1
IgG titres

| | group 1 | group 2 | group 3 | group 4 | Group 5 |
|---|---|---|---|---|---|
| T = 0 | <4.3 | <4.3 | <4.3 | <4.3 | <4.3 |
| T = 35 | 11.2 | 4.86 | 9.99 | 4.3 | 4.19 |
| T = 49 | 9.56 | 4.64 | 8.81 | 4.71 | 3.97 |
| T = 64 | 8.48 | 4.3 | 7.56 | 4.3 | 3.31 |

As depicted in Table 2 all immunised animals, including the animals in Group 2 that showed no significant anti-DON IgG titre increase, showed a significant higher weight gain during the first 15 days compared to the challenge animals. With respect to the challenged animals, all animals gained more weight over the course of the study.

TABLE 2
weight analysis

| | | | | | Average additional |
|---|---|---|---|---|---|
| | | | | | weight gain compared |
| | | | weight | weight | to challenge animals |
| | ADG1¹ | ADG² | begin | end | (grams) |
| group 1 | 0.67 | 0.80 | 11.63 | 32.29 | +1060 |
| group 2 | 0.64 | 0.79 | 12.31 | 32.13 | +760 |
| group 3 | 0.58 | 0.82 | 12.88 | 32.25 | +310 |
| group 4 | 0.54 | 0.81 | 12.69 | 31.75 | 0 |
| group 5 | 0.57 | 0.80 | 11.63 | 31.08 | +390 |

¹average daily weight gain over the first 15 days of the challenge
²average daily weight gain over the last 13 days of the challenge

The condition of the small intestines (as determined by the villus/crypt ratio in the jejunum) was also monitored. In table 3 the villus/crypt ratio is depicted. As can be seen, the animals in group 3 had an average villus crypt/crypt ratio comparable to the healthy controls (group 5), while the non-immunised, challenged group (group 4) had a much lower (statistically significant) villus crypt ratio. In addition, group 1 and group 2, had a villus/crypt ratio which was significantly better (i.e. higher) compared to the non-immunised challenge control group. This indicates that the immunisation protects against the damage of the intestine, initiated by DON.

TABLE 3
villus/crypt ratio

| | group 1 | group 2 | group 3 | group 4 | group 5 |
|---|---|---|---|---|---|
| average | 1.57 | 1.41 | 1.78 | 1.09 | 1.71 |
| STD | 0.24 | 0.22 | 0.12 | 0.10 | 0.23 |

The general condition of other organs was also monitored, more specifically the liver, the kidneys and the stomach. It was observed that all three test groups (groups 1-3) were in better health than the non-immunised challenge control group (group 4). In table 4 a summary of the general health data is depicted. The degree of stomach ulcer is reported from − (no prove of ulcer formation) to ++(multiple ulcers). The degree of stomach inflammation is reported from − (no prove of inflammation) to ++/−(initiation of stomach inflammation).

TABLE 4
General health data

| | | | Stomach | |
|---|---|---|---|---|
| | Liver colour | Stomach ulcer | inflammation | Kidneys |
| Group 1 | Normal-yellow | − | − | Pail |
| Group 2 | Normal | +/−− | − | Normal |
| Group 3 | Normal | +/− | +/−− | Normal |
| Group 4 | Pail | ++ | ++/− | Pail |
| Group 5 | Normal | + | ++/− | Normal |

### Example 2: Effect of Immunisation on DON Levels

Objective

The objective of this study was to evaluate the effects of immunization with a DON conjugate on the toxicokinetics of DON ingestion. To examine this, pigs were immunised twice with DON-KLH before being fed toxic DON.

Study Design

Ten 3 week old pigs were used in the study, divided over 2 groups of 5 pigs each. The pigs in Group 1 were immunised IM twice at 3 and 6 weeks of age with DON-KLH (Test Article 1; example1). Group 2 served as a control and only received a control fluid. At the age of 11 weeks the animals were each administered DON (Fermentek, Israel) via a bolus at a dose of 0.05 mg/kg which (based on the daily feed intake) resembled a contamination level of 1 mg/kg feed. Blood samples of the pigs were taken juts before DON administration and 0.25, 0.5, 0.75, 1, 1.5, 2, 3, 4, 6, 8, and 12 h post DON administration.

Inclusion Criteria

Only healthy animals were used.

Analysis of DON in Plasma

Plasma analysis of unbound DON was done using a validated LC-MS/MS method on an Acquity® UPLC system coupled to a Xevo® TQ-S MS instrument (Waters, Zellik, Belgium). The lower limit of quantification of DON in pig plasma using this method is 0.1 ng/ml.

Toxicokinetic Analysis

Toxicokinetic modeling of the plasma concentration-time profiles of DON was done by noncompartmental analysis (Phoenix, Pharsight Corporation, USA). Following parameters were calculated: area under the curve from time zero to infinite (AUC_{0→∞}), maximal plasma concentration (Cₘₐₓ), and time at maximal plasma concentration (tₘₐₓ).

Results

The toxicokinetic results are indicated in table 5 here beneath. As can be seen immunisation with DON-KLH decreases all toxicokinetic parameters. As it is unbound DON that is responsible for the exertion of toxic effects, it may be concluded that immunisation with DON-KLH will reduce the toxic effects caused by DON by reducing the amount of unbound DON in the blood of animals.

TABLE 5
Toxicokinetic parameters of unbound DON

| Toxicokinetic parameter | DON-KLH | Control |
|---|---|---|
| AUC_{0→∞} | 77.3 ± 23.6 | 187 ± 33 |
| Cₘₐₓ | 12.5 ± 2.7 | 30.8 ± 2.5 |
| tₘₐₓ | 1.69 ± 1.03 | 2.19 ± 1.07 |

### Example 3: Serological Response Against Various DON Conjugates

Objective

The objective of this study was to evaluate the efficacy of different conjugated deoxynivalenol products.

Study Design

Eighteen 3 week old pigs were used in the study, divided over 3 groups of six pigs each. The pigs of group 1 were immunised twice intramuscularly at 3 and 5 weeks of age with DON-KLH (using Test Article 1 of Example 1). Group 2 was immunised correspondingly with DON-OVA. Group 3 served as a negative control. All animals were checked for an anti-DON IgG response at 3 weeks of age, 5 weeks of age and 8 weeks of age.

Results

The serological results are indicated here below in the table in log 2 antibody titre.

TABLE 6
anti-DON IgG response

| Test Article | 3 weeks | 5 weeks | 8 weeks |
|---|---|---|---|
| DON-KLH | 3.5 | 6.6 | 8.3 |
| DON-OVA | 3.3 | 3.9 | 11.8 |
| Control | 4.8 | 3.3 | 3.3 |

It appears that both conjugates are suitable to raise an anti-DON IgG response. Also, a response appears be induced by one shot only.

### Example 4: Serological Response Against DON Conjugate in Chickens

Objective

The objective of this study was to evaluate the serological response of DON-KLH in chickens.

Study Design

For this study 30 four week-old chickens were used, divided over three groups of 10 chickens each. The chickens were immunized intramuscularly with DON-KLH. Group 1 was used as a control and received PBS only. Group 2 received DON-KLH without any adjuvant and group 3 received DON-KLH formulated in GNE adjuvant (available from MSD Animal Health, Boxmeer). A prime immunization was given on day 0 with 0.5 ml vaccine into right leg. On day 14, chickens received a comparable booster immunization into the left leg.

Blood sampling took place at day 0 and 14, as well as on day 35, 56, 70 and 84. Serum was isolated for the determination of IgY against DON. At day 0 and 14 blood samples were isolated just before immunisation.

Results

The serological results are depicted in table 7 in log 2 antibody titre. The PBS background has been subtracted from the data.

TABLE 7
anti-DON IgY response

| Vaccine | Day 0 | Day 14 | Day 35 | Day 56 | Day 70 | Day 84 |
|---|---|---|---|---|---|---|
| DON-KLH | 0 | 0 | 0.6 | 1.2 | 1.1 | 1.2 |
| DON-KLH in GNE | 0 | 1.9 | 6.5 | 6.0 | 6.7 | 7.7 |

As can be seen, the conjugated DON also induces an anti-DON titre in chickens. GNE adjuvant increases the response substantially but appears to be not essential for obtaining a net response as such.

### Example 5: Serological Response Against AFB Conjugate in Swine

Objective

The aim of this experiment was to assess whether or not the use of conjugated AFB in a vaccine can induce antibodies against aflatoxin in vaccinated swine.

Study Design

The vaccine contained Aflatoxin 1 (AFB1) conjugated to Bovine Serum Albumin (BSA). The conjugate was mixed with a mineral oil-containing adjuvant (XSolve 50) at a final concentration of 50 μg/ml, and applied by intramuscular (IM) administration. In the experiment, 2 groups of 6 animals were used at three weeks of age. Group one received a PCV vaccine, Porcilis® PCV (as a negative control) and Group 2 the AFB1-BSA vaccine. All primes were at three weeks of age and the boosters were at seven weeks of age. The animals were monitored for 12 weeks after start of the study.

Results

All pigs were serologically negative for titres against AFB1 at the start of the experiment (i.e. a titre of 3.5 or below). Titres developed as indicated here below In Table 8.

TABLE 8
IgG titres against AFB1

| Group | T = 7 weeks | T = 10 weeks | T = 12 weeks |
|---|---|---|---|
| 1 | 3.5 | 3.7 | 4.6 |
| 2 | 3.7 | 8.1 | 6.8 |

There appeared to be a slight titre increase in the negative control group, possibly induced by AFB1 in the feed. The titres in the conjugated AFB group however rose significantly stronger, showing a good induction of an immune response against AFB in these swine.

### Example 6: Serological Response to AFB1 in Chickens

Objective

The objective of this study was to evaluate the serological response to AFB1-KLH in chickens.

Study Design

For this study 30 four week-old chickens were used, divided over three groups of 10 chickens each. The chickens were immunized intramuscularly with AFB1-KLH. Group 1 was used as a control and received PBS only. Group 2 received AFB1-KLH without any adjuvant and group 3 received AFB1-KLH formulated in GNE adjuvant (available from MSD Animal Health, Boxmeer). A prime immunization was given on day 0 with 0.5 ml vaccine into right leg. On day 14, chickens received a comparable booster immunization into the left leg.

Blood sampling took place at day 0 and 14, as well as on day 35, 56, 70 and 84. Serum was isolated for the determination of IgY against AFB1. At day 0 and 14 blood samples were isolated just before immunisation.

Results

The serological results are depicted in table 9 in log 2 antibody titre. The PBS background has been subtracted from the data.

TABLE 9
Anti-AFB1 IgY response

| Vaccine | Day 0 | Day 14 | Day 35 | Day 56 | Day 70 | Day 84 |
|---|---|---|---|---|---|---|
| AFB1-KLH | 0.0 | 0.9 | 1.9 | 0.2 | 0.4 | 0.6 |
| AFB1-KLH in GNE | 0.0 | 5.5 | 5.8 | 4.3 | 4.0 | 4.4 |

As can be seen, the conjugated AFB1 induces an anti-AFB1 titre in chickens. GNE adjuvant increases the response substantially but appears to be not essential for obtaining a net response as such.

### Example 7: Protective Effect of AFB1 Vaccination

An in vitro potency test was performed to establish the protective effect of the serum of vaccinated animals. In this test C6 cells (glioma cell line from rats) were used, and the CCK8 (Cell counting kit 8; Dojindo Laboratories) was used to measure the viability of the cells as a response to aflatoxin. For this test it was established, via a dose response curve, that Aflatoxin 1 is toxic on these cells starting from a concentration of 20 μg/ml. In the assay as described here beneath, a concentration of 80 μg/ml was selected to assess whether reduction of the toxic effect could be accomplished by the use of serum of vaccinated animals.

For this assay C6 cells were seeded in a 96 plate, and the wells (all with the same density form the same stock) were incubated with 80 μg/ml Aflatoxin, alone, in combination with the serum of pigs vaccinated with Aflatoxin B1-KLH conjugate or in combination with serum from sero-negative animals for AFB1. All the sera were heat inactivated at 56° C. for one hour. It was observed that the positive serum incubations resulted in a higher OD450 value (corresponding to the presence of live cells) compared to the negative serum. This shows that the positive serum increased the viability of the cells and thus, that the AFB1 antibodies raised in the pigs by vaccination are able to neutralise the toxicity of AFB1 on the cells.

### Example 8: Serological Response to AFB1 in Fish

Objective

The objective of this study was to evaluate the serological response to AFB1-KLH in fish (Tilapia; Oreochromis sp).

Study Design

For the experiment a total of 100 tilapia fish were used (weighing on average 20 g), divided into two groups of fifty fish. The first group was injected intraperitoneally (IP) with 0.05 ml an AFB1-KLH mycotoxin vaccines in GNE adjuvant. The AFB1-KLH was present at a final concentration of 12.5 μg per dose. The second group was injected with standard vaccine dilution buffer (SVDB) to serve as a negative control. The fish were observed for three weeks and were then given a booster immunisation using the same vaccines as used for primary vaccination. All vaccinated fish were observed for another 2 weeks before blood sampling.

Results

The serological results are depicted in table 9 in log 2 antibody titre.

TABLE 9
Anti-AFB1 IgM response

| Vaccine | T = 2 weeks after booster |
|---|---|
| AFB1-KLH | 16.6 |
| Neg. control | <1.7 |

As can be seen, the conjugated AFB1 induces an anti-AFB1 titre in the fish. In view of the results of example 7, it is believed that the fish are protected against an AFB1 challenge.

### Example 9: Protective Effect of AFB1 Vaccination

In line with Example 7, an in vitro potency test/neutralization assay was performed with the sera raised as described in Example 8. For this, C6 cells were seeded at 2.0*10⁴ cells/ml, 100 μl per well, and grown at 37° C., 5% CO₂ for 3 days. Cells were incubated for 48 h, with a combination of 20 μg/ml AFB1 and 32× diluted serum derived from fish vaccinated with AFB1-KLH in GNE or vaccinated with a placebo. Percentages of live cells were determined by microscopical evaluation.

Serum from fish vaccinated with AFB1-KLH in GNE contains antibodies against AFB1 (see Example 8) and appeared to be protected against AFB1 damage in C6 cells. Serum from fish vaccinated with placebo did not contain antibodies against AFB1 (see Example 8) and did not protect against AFB1 damage.

TABLE 10
Percentage live cells after incubation
with AFB1 and diluted fish serum

| | Positive serum | Negative serum |
|---|---|---|
| AFB1 | 95 | 65 |

1. A method of actively protecting an animal against aflatoxin B (AFB) induced mycotoxicosis comprising administering to the animal a conjugated AFB.

2. The method according to claim 1, wherein the conjugated AFB is systemically administered to the animal.

3. The method according to claim 2, wherein the conjugated AFB is administered intramuscularly, orally and/or intradermally.

4. The method according to claim 1, wherein the conjugated AFB is administered to the animal at an age of 6 weeks or younger.

5. The method according to claim 4, wherein the conjugated AFB is administered to the animal at an age of 4 weeks or younger.

6. The method according to claim 5, wherein the conjugated AFB is administered to the animal at an age of 1-3 weeks.

7. The method according to claim 1 wherein the conjugated AFB is administered to the animal at least twice.

8. (canceled)

9. (canceled)

10. (canceled)

11. (canceled)

12. (canceled)

13. The method according to claim 1, characterised in that the animal is a swine or chicken.

14. A vaccine comprising conjugated AFB, an adjuvant and a pharmaceutically acceptable carrier.

15. The vaccine of claim 14, wherein the adjuvant is an emulsion of water and oil.

16. The vaccine of claim 15, wherein the adjuvant is a water-in-oil emulsion or an oil-in-water emulsion.

17. The vaccine of claim 14, wherein the conjugated AFB comprises AFB conjugated to a protein having a molecular mass above 10.000 Da.

18. The vaccine of claim 14, wherein the conjugated AFB comprises AFB conjugated to keyhole limpet hemocyanin (KLH) or ovalbumin (OVA).
